(19) United States
(12) Patent Application Publication
(10) Pub. No.: US 2020/0088740 A1
(43) Pub. Date: Mar. 19, 2020

(54) METHOD TO DETERMINE IF A RETINA IS DAMAGED

(71) Applicant: Zhongshan Ophthalmic Center, Sun Yat-sen University, Guangzhou (CN)

(72) Inventor: Zhongshu Tang, Guangzhou (CN)

(73) Assignee: Zhongshan Ophthalmic Center, Sun Yat-sen University

(21) Appl. No.: 16/403,133

(22) Filed: May 3, 2019

(30) Foreign Application Priority Data
Sep. 17, 2018 (CN) 201811084157.1

(51) Int. Cl. G01N 33/68; G01N 33/564

(52) U.S. Cl. CPC G01N 33/6893; A61B 3/16; G01N 33/564

(57) ABSTRACT

This invention provides a test for retina lesions. It aims to test whether the retina is damaged by determining whether there are synaptic vesicle proteins in the aqueous humour. The synaptic vesicle proteins include synaptic vesicle protein 2. This invention proposes to determine whether a retina is damaged by determining whether there is synaptic vesicle protein 2 in the aqueous humour. It provides a method to decide the degree to which a retina is damaged in clinic. It is useful to predict the transition from ocular hypertension to glaucoma, so as to cure and preclude glaucoma.

### BACKGROUND

### Technical Field

This invention is to predict whether retinae are damaged to an extent that patients will suffer from glaucoma in the future.

### Background

Aqueous humour circulates in the eyes to retain their normal function. Aqueous humour is created in the ciliary body and flows out of the eyes via the trabecular meshwork. Any defect that affects the aqueous humour circulation, either to increase its creation or decrease its outflow, will increase intraocular pressure and possibly further lead to glaucoma.

Retina damage is the result of ocular hypertension, and may cause glaucoma. But ocular hypertension does not always lead to glaucoma. And furthermore, the transition from ocular hypertension to glaucoma takes years. Currently there is no way to determine whether ocular hypertension will lead to glaucoma or not in clinical practice, so overtreatment is common in glaucoma clinical practice. A method that can determine the transition of ocular hypertension to glaucoma is desired.

Current methods for examining glaucoma include:

1) Campimetry is one way to systematically test the visual field. Visual field loss is a feature of glaucoma;

2) Optical Coherence Tomography (OCT) measures the thickness of retinal nerve fiber layer. Decrease of the thickness is also a feature of glaucoma; and

3) Fundus images that show the morphology of optic nerve ahead and allow a calculation of the cup-disc-ratio. Increase of cup-disc-ratio is another feature of glaucoma.

None of the above methods can predict the occurrence of glaucoma.

### SUMMARY

A method to determine retinal damage caused by ocular hypertension, to determine whether an aqueous humour contains at least a synaptic vesicle protein for the prediction of whether the ocular hypertension will develop into glaucoma.

### DESCRIPTION OF THE DRAWINGS

In the drawings:

FIG. 1 depicts terminal deoxynucleotidyl transferase TdT-mediated dUTP nick end labeling (TUNEL) staining of apoptotic cells in ocular hypertension in accordance with one embodiment of the disclosure;

FIG. 2 depicts leakage of synaptic vesicle protein 2 (SV2) out of retina damaged caused by ocular hypertension in accordance with one embodiment of the disclosure;

FIG. 3 is an example scheme of the ocular hypertension animal model in accordance with one embodiment of the disclosure; and

FIG. 4 is an example dot blot assay of SV2 in aqueous humour of individual mice in accordance with one embodiment of the disclosure.

### DETAILED DESCRIPTION OF THE INVENTION

The embodiments listed below are written only to illustrate the applications of this apparatus and method, not to limit the scope. The equivalent form of modifications towards this apparatus and method shall be categorized as within the scope the claims.

Certain terms are used throughout the following description and claims to refer to particular system components. As one skilled in the art will appreciate, different companies may refer to a component and/or method by different names. This document does not intend to distinguish between components and/or methods that differ in name but not in function.

In the following discussion and in the claims, the terms “including” and “comprising” are used in an open-ended fashion, and thus may be interpreted to mean “including, but not limited to . . . .” Also, the term “couple” or “couples” is intended to mean either an indirect or direct connection. Thus, if a first device couples to a second device that connection may be through a direct connection or through an indirect connection via other devices and connections.

None of the methods currently available in clinic can predict a transition from ocular hypertension to glaucoma. This invention provides a method to determine whether a retina is damaged by ocular hypertension to an extent that results in glaucoma in the fixture. That is, to test whether there are synaptic vesicle proteins in the aqueous humour of a patient. If there are, the patient may suffer glaucoma in the future.

For that purpose, this invention provides a method to determine whether the retina is damaged. One aspect of the present application is a method to determine the existence of synaptic vesicle proteins in aqueous humour as a prediction of glaucoma.

In one embodiment, the method comprises the step of checking synaptic vesicle protein 2 in the aqueous humour.

This method described proposes to determine whether there are synaptic vesicle proteins in the aqueous humour using antibodies against synaptic vesicle proteins.

In one embodiment, this method proposes to determine whether there is synaptic vesicle protein 2 in the aqueous humour using an antibody against synaptic vesicle protein 2.

This method proposes to determine whether there is synaptic vesicle protein 2 in the aqueous humour by the methods that include but are not limited to dot blot, electrophoresis, dipsticks and ELISA (enzyme linked immunosorbent assay).

Compared to current methods, this invention provides methods to determine whether a retina is damaged by checking whether there are synaptic vesicle proteins in the aqueous humour of a patient. This invention is a useful application in clinical practice to predict whether a retina is damaged sufficiently by ocular hypertension that it will result in glaucoma in the future.

### Methods

This invention is to test whether a retina is damaged by ocular hypertension.

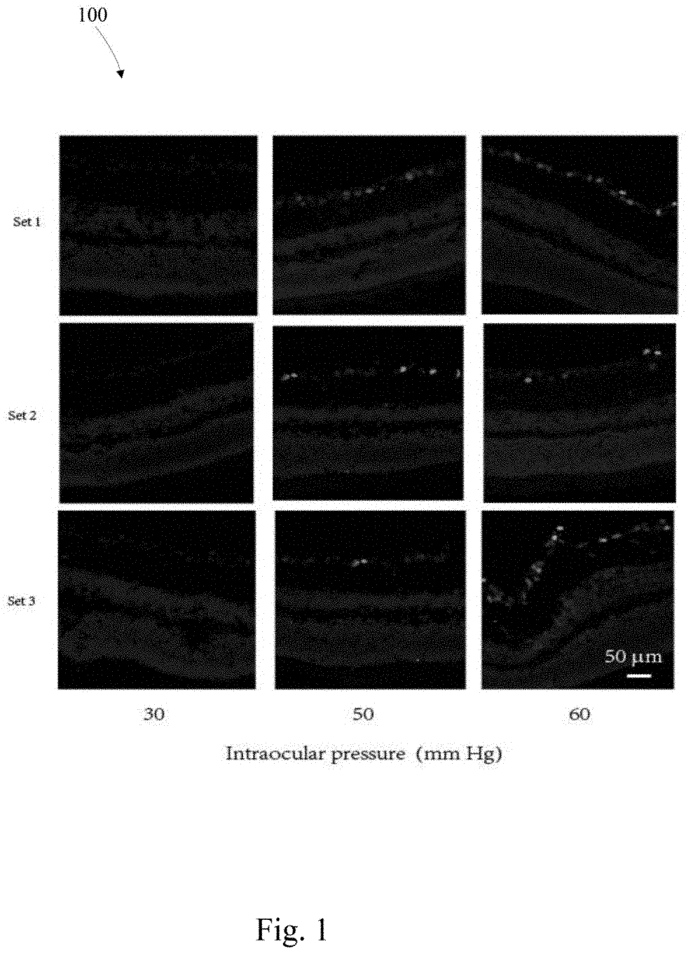

The following methods in FIGS. 1 to 4 describe protocols that test whether a retina is damaged by ocular hypertension.

Terminal deoxynucleotidyl transferase TdT-mediated dUTP nick end labeling (TUNEL) is a method to label apoptotic cells in biomedical research. It uses the terminal deoxynucleotidyl transferase (TdT) to catalyze the attachment of fluorochrome-tagged deoxynucleotides to the 3′-hydroxyl termini of double strand DNA breaks. The fluorescence-labeled TUNEL+ cells can be observed under a microscope.

Under an ocular hypertension animal model we detected many TUNEL+ apoptotic cells which indicated that the retina was damaged. As shown in FIG. 1, no TUNEL+ cells were detected under 30 mm Hg. But under the intraocular pressure of 50 and 60 mm Hg, there were TUNEL+ apoptotic cells which appear as white in FIG. 1.

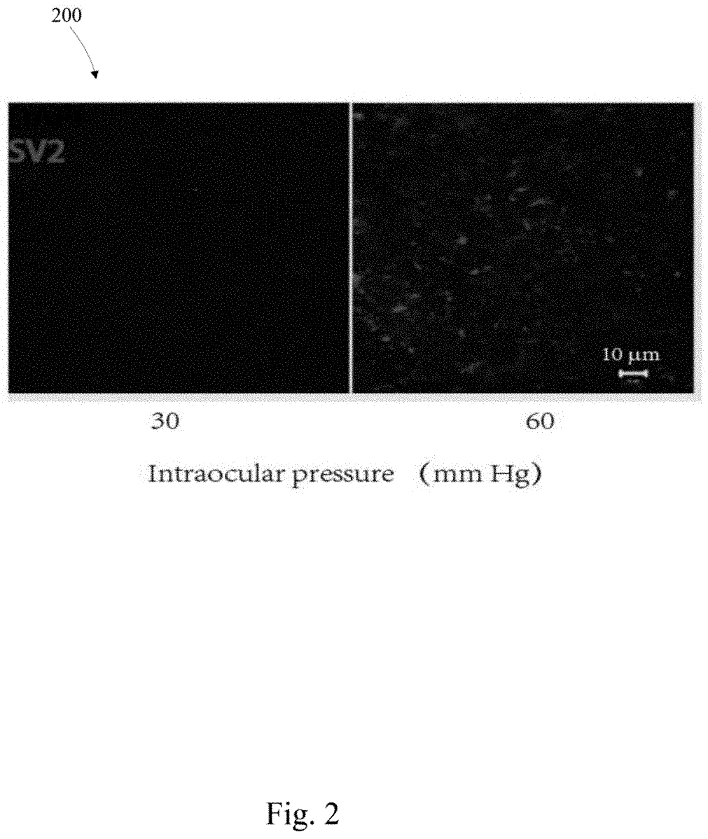

We also did immunostaining using an antibody against synaptic vesicle protein 2 under the condition of non-permeabilization in which extracellular proteins can be visualized in a whole-mount retina. As shown in the left panel in FIG. 2, there was no SV2 under 30 mm Hg treatment.

But under the pressure of 40 mm Hg or above, as shown in the right panel of FIG. 2, SV2 was observed in the extracellular space which was supposed to be released from neurons which were damaged by ocular hypertension.

Synaptic vesicle protein 2 is an intracellular protein and normally is only discovered inside neurons. The extracellular existence of SV2 indicated that the integrity of neuronal membrane was damaged, so that the retina was damaged.

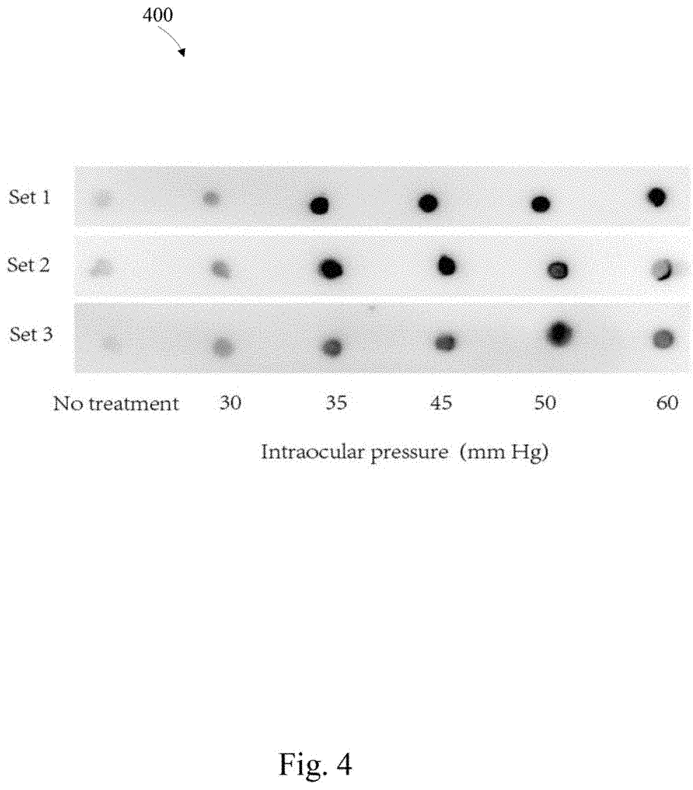

Further study indicated that synaptic vesicle protein 2 can be found in the aqueous humour of eyes that were damaged by ocular hypertension (FIG. 4). As aqueous humour can be obtained in a simple clinical procedure, it is a simple and effective way to determine the damage of a retina by determining the existence of synaptic vesicle protein 2 in the aqueous humour.

### Protocols

1. Ocular Hypertension Mouse Model.

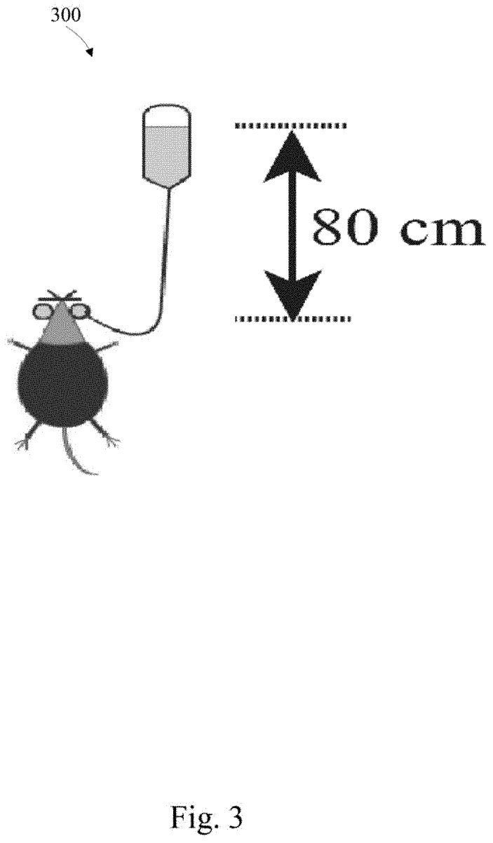

Animal experiments in this study were handled in accordance with the guideline approved by the Institutional Animal Care and Use Committee of Zhongshan Ophthalmic Center (2015-023). An acute ocular hypertension animal model was established in the following way (FIG. 3). Briefly, mice were anaesthetized by i.p. injection of chloral hydrate and the corneas were topically anaesthetized with 0.5% tetracaine hydrochloride. Pupils were dilated with 1% tropicamide. A 30-gauge infusion needle was cannulated into the anterior chamber of one eye to connect with a normal saline reservoir which was elevated to the height as required, e.g., 80 cm for a pressure of 60 mm Hg. At the end of the hypertension, the reservoir was lowered to the height of the treated eye and the needle was withdrawn.

2. TUNEL Staining

Three days after the ocular hypertension treatment, the animals were anaesthetized and perfused with PFA via cardial ascending aorta. A whole mount retina was prepared for TUNEL staining of apoptotic cells. Briefly, whole-mount retinae or cryostat sections were permeabilized and incubated with TUNEL reaction mixture (In situ Cell Death Detection kit; Roche Applied Science, USA) for 60 min at 37° C. After PBS wash, the samples were incubated in 0.1% DAPI for 5 min followed by washing in PBS, and examined under a confocal microscope (LSM710; Carl Zeiss) with the same imaging parameters.

3. Immunofluorescence Staining of SV2

After perfusion with PFA, the fixed eye ball was taken out. Whole mount retina was prepared, and proceeded for immunofluorescence staining of SV2.

Wash the retina 3 times, with 10 min each under shake.

Immerse the retina under PBS buffer containing 5% donkey serum for 1 hr.

Incubate the retina under PBS buffer containing 5% donkey serum and mouse-anti-SV2 primary antibody (1:200) for overnight with shake.

Wash the retina 3 times, with 10 min each under shake.

Incubate the retina under PBS buffer containing 5% donkey serum and Alexa 488-donkey-anti-mouse secondary antibody (1:10,000) for 1 hr under room temperature.

Wash the retina 3 times, with 10 min each under shake.

Incubate the retina under DAPI solution for 5 min.

Cut the retina into a flower with 4 petals, and mount it on a slide glass, cover with a coverslip. Get ready for observation under fluorescent microscope.

4. Western Blot of SV2 in Aqueous Humour

Mice were treated with 2 hr of hypertension and still 5 hr after the release of hypertension, aqueous humour were collected directly from the anterior chamber.

A piece of PVDF membrane was marked with lattice which indicates the position for dot.

Three microliters of each sample were dotted a each spot.

The membrane was dried under room temperature for over 1 hr.

Wash the membrane shortly. And incubate it with TBS buffer containing 5% milk for 1 hr.

Incubate the membrane under TBS buffer containing 5% milk and mouse-anti-SV2 primary antibody (1:500) for overnight with shake.

Wash the membrane 3 times, with 5 min each under shake.

Incubate the membrane under TBS buffer containing 5% milk and HRP-donkey-anti-mouse secondary antibody (1:10,000) for 1 hr under room temperature.

Wash the r membrane 3 times, with 10 min each under shake.

The antibody bound membrane was visualized by enhanced chemiluminescence Western blotting detection reagents (Millipore, Billerica, Mass., USA), and further exposed with a cool-CCD image system.

FIG. 1. Depicts a TUNEL staining of apoptotic cells ocular hypertension. The strong white color indicates TUNEL+ apoptotic cells.

FIG. 2. Depicts a leakage of SV2 out of retinae damaged by ocular hypertension. The left panel indicates the condition of 30 mmHg which does not induce damage yet. The right panel indicates the condition of 40 mmHg in which damage does occur so that SV2 (white) was deposited extracellularly.

FIG. 3. Depicts a scheme of an ocular hypertension animal model. An infusion needle was cannulated into the anterior chamber of one eve to connect it with reservoir which was elevated to the height as required to 80 cm for a pressure of 60 mm Hg.

FIG. 4. Depicts a dot blot assay of SV2 in aqueous humour of individual mice. Aqueous humour sample from single individuals were dotted on spots of a PVDF membrane. After going-through a process of immuno-labeling, the membrane was visualized by a digital camera system. The black dots indicate the existence of SV2. The gray color in “no treatment” or “30 mm Hg” groups indicates background of non-specific proteins.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code may be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

The word “example” is used herein to mean “serving as an example or illustration.” Any aspect or design described herein as “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

References to “one embodiment,” “an embodiment,” “some embodiments,” “various embodiments”, or the like indicate that a particular element or characteristic is included in at least one embodiment of the invention. Although the phrases may appear in various is places, the phrases do not necessarily refer to the same embodiment. In conjunction with the present disclosure, those skilled in the art will be able to design and incorporate any one of the variety of mechanisms suitable for accomplishing the above described functionalities.

It is to be understood that the disclosure teaches just one example of the illustrative embodiment and that many variations of the invention can easily be devised by those skilled in the art after reading this disclosure and that the scope of then present invention is to be determined by the following claims.

What is claimed is:

1. A method to determine retinal damage caused by ocular hypertension, comprising, determining whether an aqueous humour contains at least a synaptic vesicle protein.

2. The method to determine retinal damage caused by ocular hypertension of claim 1, wherein the synaptic vesicle protein contains at least a synaptic vesicle protein 2.

3. The method to determine retinal damage caused by ocular hypertension of claim 1, wherein an antibody of the synaptic vesicle protein is utilized to determine whether the aqueous humour contains the synaptic vesicle protein.

4. The method to determine retinal damage caused by ocular hypertension of claim 2, wherein an antibody of the synaptic vesicle protein 2 is utilized to determine whether the aqueous humour contains the synaptic vesicle protein 2.

5. The method to determine retinal damage caused by ocular hypertension of claim 4, wherein a response to the antibody of the synaptic vesicle protein 2 is determined based on at least one of a dot blot, an electrophoresis, a dipstick and an enzyme linked immunosorbent assay.
